(19) United States
(12) Patent Application Publication
(10) Pub. No.: US 2025/0331758 A1
(43) Pub. Date: Oct. 30, 2025

(54) METHODS FOR ACHIEVING AN ISOLATED ELECTRICAL INTERFACE BETWEEN AN ANTERIOR SURFACE OF A MICRONEEDLE STRUCTURE AND A POSTERIOR SURFACE OF A SUPPORT STRUCTURE

(71) Applicant: Biolinq Incorporated, San Diego, CA (US)

(72) Inventors: Joshua WINDMILLER, San Diego, CA (US); James Patrick MCCANNA, San Diego, CA (US); Jared Rylan TANGNEY, Encinitas, CA (US)

(21) Appl. No.: 19/258,472

(22) Filed: Jul. 2, 2025

Related U.S. Application Data
(63) Continuation of application No. 18/824,598, filed on Sep. 4, 2024, now Pat. No. 12,369,830.
Application No. 18/824,598 is a continuation of application No. 15/913,709, filed on Mar. 6, 2018, now Pat. No. 12,109,032.
(60) Provisional application No. 62/470,204, filed on Mar. 11, 2017.

(51) Int. Cl. A61B 5/296; A61B 5/00; A61B 5/1486

(52) U.S. Cl. CPC A61B 5/296; A61B 5/14865; A61B 5/6848; A61B 5/685

(57) ABSTRACT

A method for circumscribing an insulating barrier region around a singular conductive microneedle structure or plurality of conductive microneedle structures adhered to a fixed substrate for the purpose of spatially defining a conduit for the routing of an electrical signal from the surface of said microneedle or microneedles to the posterior surface of the substrate is disclosed herein. A microneedle-based electrochemical biosensors structure comprises a substrate, a microneedle biosensor, a primary electrically conductive element, a secondary electrically conductive element and an electrically insulative annular barrier.

### CROSS REFERENCE TO RELATED APPLICATION

The present Application claims priority to U.S. Provisional Patent Application No. 62/470,204 filed on Mar. 11, 2017, which is hereby incorporated by reference in its entirety.

### STATEMENT REGARDING FEDERALLY SPONSORED RESEARCH OR DEVELOPMENT

Not Applicable

### BACKGROUND OF THE INVENTION

### Field of the Invention

The present invention generally relates to methods for manufacturing microneedle structures.

### Description of the Related Art

Microneedle-based electrochemical biosensor devices have witnessed increased development activity in recent years and rely on the interaction between an electrical signal and a biological analyte. However, to serve useful purposes, these devices must be fabricated using design topologies that are compatible with existing packaging methods. In this vein, current chip-scale implementations of microneedle biosensors (and drug delivery actuators, for that matter) require wire-bonding or flip-chip methods of establishing electrical contact with the active sensing (or transducing) element located on or within the microneedle structure. In spite of this approach, wire bonding and flip-chip methods are not compatible with useful embodiments of sensing/therapeutic capabilities as these modalities would serve to obstruct the microneedle surface and prevent reliable penetration of the stratum corneum. The current methodology allows for an alternative to wire-bonding and flip-chip methods of electrical interface by means of circumscribing electrically-conductive microneedles with an insulating barrier to facilitate a spatially-defined region that can be probed on the posterior surface of the substrate on which the microneedle elements are located.

Microneedle-based electrochemical biosensors represent a promising avenue towards the minimally-invasive quantification of a number of relevant analytes in the physiological fluid, such as interstitial fluid, blood, serum, and plasma. In such devices, an electrical signal is applied to the surface of a microneedle, which concomitantly instigates an electrochemical reaction to either oxidize or reduce an analyte or product of the analyte and a surface-immobilized intermediary. The resultant magnitude of potential or current arising from the redox reaction, which reflects the concentration of analyte in the physiological medium, is then routed to an external integrated circuit, module, or self-contained device for interpretation. In order to facilitate electrical interface between the skin-penetrating regions of the microneedle and a suitable location where the redox signal can be probed, conductive traces are spatially defined from the sensing region located on the microneedle surface to a pad located on the extremities of the substrate upon which the microneedles are secured. However, current chip-scale packaging techniques such as wire-bonding mandate that electrical leads be soldered on the same surface in which the microneedles are located; this can interfere with the insertion of the said microneedles due to increased standoff. On the other hand, flip-chip techniques mandate that the substrate be ‘flipped’ to facilitate electrical interface, which thereby serves to orient the microneedle structure(s) away from the intended application site and into the interior of the package itself. In order to maintain compatibility with existent packaging methods, the fabrication of said microneedle devices mandates that a spatially-defined conductive region be realized between the sensing surface located on the microneedle structure and the posterior surface of the substrate that the said microneedle is located. In accordance with current practice, this has proven difficult to remedy as the spatial definition of a suitable conduit providing electrical communication between two opposing sides of the substrate required modifications to the fabrication methodology.

Prior art solutions have been concerned with patterned, planar conductive traces routing the electrical signal from the microneedle surface to a spatially defined region (pad) suitable for probing/wire-bonding to facilitate implementation in an integrated circuit-style package. The said pad is located on the same surface as the microneedle structure, which, when attached to an external circuit with bonding wire, will cause increased standoff and prevent the microneedle structure from fully inserting into the viable epidermis or dermis. The trace is not routed through the substrate supporting said microneedle(s) and occupies the same surface as the microneedle.

U.S. Pat. No. 6,551,849 for a Method For Fabricating Arrays Of Micro-Needles discloses an array of micro-needles is created by forming an array pattern on the upper surface of a silicon wafer and etching through openings in the pattern to define micro-needle sized cavities having a desired depth. The mold thus formed may be filled with electrically conductive material, after which a desired fraction of the silicon wafer bulk is removed from the bottom-up by etching, to expose an array of projecting micro-needles. The mold may instead be filled with a flexible material to form a substrate useful in gene cell probing. An array of hollow micro-needles may be formed by coating the lower wafer surface with SiN, and etching through pattern openings in the upper surface down to the SiN layer, and then conformally coating the upper surface with thermal silicon dioxide. The SiN layer is then stripped away and a desired fraction of the bulk of the wafer removed from the bottom-up to expose an array of projecting hollow micro-needles.

U.S. Patent Publication Number 20140303471 for Non-Invasive Segmentable Three-Dimensional Microelectrode Array Patch For Neurophysiological Diagnostics And Therapeutic Stimulation discloses implementations disclosed herein provide for a microneedle electrode system comprising a microneedle electrode patch connected to external electronics. The microneedle electrode patch comprises a first flexible substrate having a plurality of conductive pads disposed thereon, a plurality of three-dimensional, individually addressable microneedle electrode arrays where each array has a plurality of microneedles extending from an upper surface thereof and a lower surface adapted to contact a corresponding one of the plurality of conductive pads disposed on the first substrate, and a second flexible substrate having a plurality of openings defined therein dimensioned to accommodate at least a portion of the upper surface of the microneedle electrode array from which the microneedles extend. Each of the conductive pads is disposed in electrical communication with a corresponding one of the plurality of microneedle electrode arrays and the first and second substrate are bonded together such that each one of the plurality of microneedle electrode arrays extends through a corresponding one of the plurality of openings defined in the second substrate.

U.S. Pat. No. 8,308,960 for Methods for Making Microneedles and Applications Thereof discloses a method of making vertically protruding elements on a substrate, said elements having a tip comprising at least one inclined surface and an elongated body portion extending between said substrate and said tip. The method comprises an anisotropic, crystal plane dependent etch forming said inclined surface(s); and an anisotropic, non crystal plane dependent etch forming said elongated body portion; combined with suitable patterning processes defining said protruding elements to have a predetermined base geometry.

U.S. Pat. No. 8,637,351 for Methods for Making Microneedles and Applications Thereof discloses a method of making vertically protruding elements on a substrate, said elements having a tip comprising at least one inclined surface and an elongated body portion extending between said substrate and said tip. The method comprises an anisotropic, crystal plane dependent etch forming said inclined surface(s); and an anisotropic, non crystal plane dependent etch forming said elongated body portion; combined with suitable patterning processes defining said protruding elements to have a predetermined base geometry.

### BRIEF SUMMARY OF THE INVENTION

The current solution teaches of a means to implement a spatially-defined, conductive conduit between the sensing surface of a microneedle (or array of microneedles) located on a planar substrate and the opposing surface of said substrate. In doing so, the need to make ohmic contact with the microneedle-containing surface of the substrate is mitigated, which enables unobstructed insertion of the said microneedle (or array of microneedles) into the viable epidermis or dermis.

The technology described herein involves a method for circumscribing an insulating barrier region around a singular conductive microneedle structure or plurality of conductive microneedle structures adhered to a fixed substrate for the purpose of spatially defining a conduit for the routing of an electrical signal from the surface of said microneedle or microneedles to the posterior surface of said substrate.

One aspect of the present invention is a microneedle-based electrochemical biosensors structure. The structure comprises a substrate, a microneedle biosensor, a primary electrically conductive element, a secondary electrically conductive element and an electrically insulative annular barrier. The substrate is composed of an electrically conducting material, the substrate comprising an anterior surface and a posterior surface. The microneedle biosensor has a length ranging from 20 microns to 2000 microns. The microneedle biosensor comprises a penetrating end and a posterior end. The microneedle biosensor protrudes from the anterior surface of the substrate and having a portion within the substrate. The primary electrically conductive element is located on the penetrating end of the microneedle biosensor. The secondary electrically conductive element is located on the posterior end of the microneedle biosensor. The electrically insulative annular barrier surrounds the portion of microneedle biosensor that is embedded in the substrate to electrically isolate the microneedle biosensor from the substrate.

Another aspect of the present invention is a microneedle-based electrochemical biosensors structure. The structure comprises a substrate, a plurality of microneedle biosensors, a primary electrically conductive element, a secondary electrically conductive element and an electrically insulative annular barrier. The substrate is composed of an electrically conducting material, the substrate comprising an anterior surface and a posterior surface. Each of the plurality of microneedle biosensors has a length ranging from 20 microns to 2000 microns. Each microneedle biosensor comprises a penetrating end and a posterior end, each microneedle biosensor protruding from the anterior surface of the substrate and having a portion within the substrate. The primary electrically conductive element is located on the penetrating end of each microneedle biosensor. The secondary electrically conductive element is located on the posterior end of each microneedle biosensor. The electrically insulative annular barrier surrounds the portion of each microneedle biosensor that is embedded in the substrate to electrically isolate the microneedle biosensor from the substrate.

Yet another aspect of the present invention is a microneedle-based electrochemical biosensors structure with a printed circuit board (PCB). The structure comprises a PCB, a substrate, a plurality of microneedle biosensors, a primary electrically conductive element, a secondary electrically conductive element, an electrically insulative annular barrier and a plurality of phase-change conductive interconnects. The PCB comprises a pad, at least one via and a plurality of traces. The substrate is composed of an electrically conducting material, the substrate comprising an anterior surface and a posterior surface. Each of the plurality of microneedle biosensors has a length ranging from 20 microns to 2000 microns. Each microneedle biosensor comprises a penetrating end and a posterior end, each microneedle biosensor protruding from the anterior surface of the substrate and having a portion within the substrate. The primary electrically conductive element is located on the penetrating end of each microneedle biosensor. The secondary electrically conductive element is located on the posterior end of each microneedle biosensor. The electrically insulative annular barrier surrounds the portion of each microneedle biosensor that is embedded in the substrate to electrically isolate the microneedle biosensor from the substrate. The phase-change conductive interconnect is positioned between the pad of the printed circuit board and the secondary electrically conductive element.

Yet another aspect of the present invention is a microneedle-based electrochemical biosensors structure with a printed circuit board (PCB). The structure comprises a PCB, a substrate, a microneedle biosensor, a primary electrically conductive element, a secondary electrically conductive element, an electrically insulative annular barrier and a plurality of phase-change conductive interconnects. The PCB comprises a pad, at least one via and a plurality of traces. The substrate is composed of an electrically conducting material, the substrate comprising an anterior surface and a posterior surface. Each of the plurality of microneedle biosensors has a length ranging from 20 microns to 2000 microns. Each microneedle biosensor comprises a penetrating end and a posterior end, each microneedle biosensor protruding from the anterior surface of the substrate and having a portion within the substrate. The primary electrically conductive element is located on the penetrating end of each microneedle biosensor. The secondary electrically conductive element is located on the posterior end of each microneedle biosensor. The electrically insulative annular barrier surrounds the portion of each microneedle biosensor that is embedded in the substrate to electrically isolate the microneedle biosensor from the substrate. Each of the phase-change conductive interconnects is positioned between the pad of the printed circuit board and the secondary electrically conductive element of each microneedle biosensor.

Yet another aspect of the invention is the jetting of solder balls, spheres, or solder bumps onto the secondary conductive element on located on the posterior end of each microneedle biosensor to facilitate solder reflow with pads located on the IC, IC package, chip carrier, or PCB. Said solder balls, spheres, or solder bumps may be simultaneously jetted onto the secondary conductive element and heated by means of a reflow laser to facilitate adhesion of said solder balls, spheres, or solder bumps onto the underlying secondary conductive element. Optionally, said process can be implemented on a wafer scale or by means of wafer-level solder balling to accelerate the rate at which said jetting occurs. In such a process, solder balls, spheres, or solder bumps are attached, simultaneously, to all the secondary conductive elements located on the posterior ends of each microneedle biosensor on a wafer substrate in a highly parallelized fashion.

Yet another aspect of the invention is the attachment of an electrically-conductive epoxy to at least one of a secondary conductive element on located on the posterior end of each microneedle biosensor and a pad located on an IC, IC package, chip carrier, or PCB. Optionally, heat can be employed to reflow the electrically-conductive epoxy. Optionally, the electrically-conductive epoxy can cure following UV exposure. Optionally, the electrically-conductive epoxy can cure following remaining at ambient conditions for a defined period of time.

Yet another aspect of the invention is the incorporation of a conducting metallic pillar into the solder ball, sphere, or bump to increase the standoff between the secondary electrically conductive element located on the posterior end of each microneedle biosensor and the underlying pad located on the surface of an IC, IC package, chip carrier, or PCB.

Yet another aspect of the invention is the selection of the secondary conductive element on located on the posterior end of each microneedle biosensor to facilitate stud bumping or the attachment of solder balls. Said conductive element can be selected from, for example, gold, nickel, aluminum, and palladium or be comprised of a bi- or tri-metallic alloy of any permutation of the same.

Yet another aspect of the invention is the selection of the pad material located on the IC, IC package, chip carrier, or PCB. The selection of the material is to facilitate stud bumping or the attachment of solder balls. Said pad material can be selected from, for example, gold, nickel, aluminum, and palladium or be comprised of a bi- or tri-metallic alloy of any permutation of the same.

Having briefly described the present invention, the above and further objects, features and advantages thereof will be recognized by those skilled in the pertinent art from the following detailed description of the invention when taken in conjunction with the accompanying drawings.

### BRIEF DESCRIPTION OF THE SEVERAL VIEWS OF THE DRAWINGS

FIG. 1 is a cross-sectional view of a singular electrically-conductive microneedle structure located on the anterior surface of an electrically-conductive substrate.

FIG. 1A is a cross-sectional view of multiple microneedle structures located on the anterior surface of an electrically-conductive substrate.

FIG. 2 is a top plan view of an anterior surface of a substrate featuring a microneedle element circumscribed by an insulating barrier.

FIG. 2A is a top plan view of an anterior surface of a substrate featuring microneedle elements circumscribed by an insulating barrier.

FIG. 3 is a view of a posterior surface of a substrate featuring a secondary electrically-conductive element (pad) circumscribed by an insulating barrier.

FIG. 4 is a preliminary phase of a fabrication process implemented to define insulating barrier regions to electrically isolate a microneedle and underlying substrate to facilitate electrical probing from the posterior surface of the substrate.

FIG. 4A is a first step of the fabrication process in which a substrate is etched from a single side.

FIG. 4B is a second step of the fabrication process in which the etched void is filled with an insulating material.

FIG. 4C is a third step of the fabrication process in which an opposing side of the substrate is etched.

FIG. 4D is a fourth step of the fabrication process in which the opposing side void is filled with an insulating material.

FIG. 5 is a cross-sectional view of singular electrically-conductive microneedle structures, electrically-isolated from one another, and located on the anterior surface of an electrically-conductive substrate.

FIG. 6 is a cross-sectional view of a prior art flip-chip bonding configuration and method of interface.

FIG. 6A is a top plan view of a prior art packaged microneedle sensing device.

FIG. 7 is a cross-sectional view of the prior art wire bonding configuration and method of interface.

FIG. 7A is a top plan view of a prior art packaged microneedle sensing device.

FIG. 8 is a cross-sectional view of two electrically-isolated microneedles occupying the same substrate to illustrate a solder ball/stud bumping configuration and method of interface.

### DETAILED DESCRIPTION OF THE INVENTION

The technology disclosed herein addresses the above challenge via the implementation of fully-insulating barriers that circumscribe and thereby electrically isolate the microneedle (or plurality of microneedles) from other locations on the substrate. In this manner, an electrical signal can be routed from a spatially-defined electrically-conductive region located on the tip of the microneedle (or plurality of microneedles) to the posterior surface of the substrate to facilitate probing and device-level packaging while leaving the surface containing the microneedle(s) exposed to enable skin penetration and subsequent sensing operation. An electrically-conductive substrate (i.e. semiconductor wafer, polymer wafer, glass wafer, ceramic wafer, or metal wafer) containing microneedles on the anterior surface (each microneedle featuring a primary electrically-conductive element (i.e. metal, semiconductor, conducting polymer) in ohmic contact with the conductive substrate) and a secondary electrically-conductive element (i.e. metal, semiconductor, conducting polymer) on the posterior surface undergoes an etching process whereby substrate material in an annular region circumscribing one or more conductive microneedles is removed. The trench created in this process, which must be less than the thickness of the substrate, is filled with an insulating material (growth of a native oxide or deposition of an electrically-insulating compound). The substrate is then flipped to expose the opposing surface and another trench (having identical geometric features to the aforementioned trench) is etched to a depth sufficient to access the opposing filled trench. This trench is subsequently filled with an insulating material (growth of a native oxide or deposition of an electrically-insulating compound). In this fashion, an isolated, electrically-conducting region extending from the primary electrical contact (located on or within the microneedle structure) to the secondary electrical contact is realized. The secondary electrically-conductive element is either probed directly, bonded to solder bumps and exposed to solder reflow to attach to pads populating a printed circuit board (PCB), or bonded to solder bumps located within an integrated circuit package. The IC package can be probed directly, placed in a socket, or soldered onto a printed circuit board to facilitate electrochemical analysis (control of the redox reaction and readout of the signal). This approach is in direct analogy to conductive through-silicon vias implemented in silicon-based semiconductor substrates, which are widely used in 3-dimensional semiconductor manufacturing and wafer-level packaging. The differentiating feature in the current innovation resides in the approach of ‘filling’ the via with insulating material and circumscribing an enclosed region with said via to facilitate full electrical isolation from other portions of the substrate not located within the enclosed region.

The structure preferably includes a substrate, a microneedle biosensor, a primary electrically-conductive element, a secondary electrically-conductive element, and an electrically-insulative annular barrier. The substrate is fabricated from an intrinsically conducting or semiconducting material (i.e. doped semiconductor wafer, conducting polymer wafer, glass wafer, ceramic wafer, or metal wafer). The substrate features anterior and posterior surfaces. The microneedle biosensor is a defined protrusion from the anterior surface of the substrate possessing vertical extent of between 20 and 2000 μm and designed to penetrate the stratum corneum or other biological tissue to access a viable physiological fluid (such as blood, plasma, serum, or interstitial fluid). The primary electrically-conductive element is a defined conductive region located on the surface of said microneedle structure, intended to react, either directly or indirectly, with a chemical analyte in the physiological medium. The secondary electrically-conductive element is a defined conductive region located on the posterior surface of said substrate, intended to enable ohmic contact with a electrical probing mechanism or bonding/soldering to an integrated circuit package or printed circuit board. The electrically-insulative annular barrier is an annular barrier, defined via an etching, milling, machining, ablative, or otherwise subtractive processes, filled with an electrically insulating material to spatially define a region of conductivity within the annulus and provide isolation with the substrate located to the exterior of said annulus. The electrically insulating material can be filled by means of the oxidative growth of a native, non-conducting oxide, nitride, or other alloy or the additive deposition of the same.

The method for manufacturing the structure includes etching, milling, machining, or ablating of an anterior trench in which an etching process is used on the anterior surface of a conductive substrate to remove material in a defined patterned region, thereby defining an anterior trench. The method also includes implementation of an insulating material in anterior trench in which an insulating layer is grown or deposition of an insulating material in said anterior trench. The method also includes etching, milling, machining, or ablating of the posterior trench in which one of the etching, milling, machining, or ablating process is used on the posterior surface of said conductive substrate to remove material in a defined patterned region, thereby defining a posterior trench. Substrate material is etched, milled, machined, or ablated to a depth that is equal to or greater than the difference between the substrate thickness and the depth of the anterior trench, but less than the overall thickness of the substrate. The method also includes implementation of an insulating material in posterior trench in which an insulating layer is grown or deposition of an insulating material in said posterior trench.

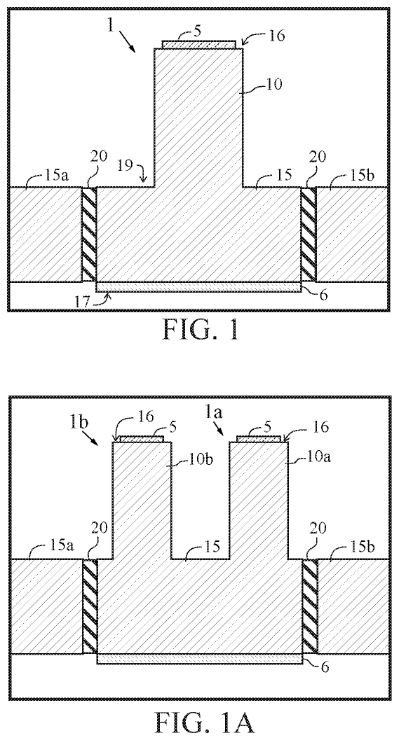

FIGS. 1 and 1A illustrate a singular electrically-conductive microneedle structure 1 and a plurality of microneedle structures 1a and 1b located on the anterior surface 16 of an electrically-conductive substrate 15. Insulating barriers 20 are shown and possess a vertical extent equal to the thickness of the substrate 15. A primary electrically-conductive sensing element 5 is located on the microneedle structure 10 and a secondary electrically-conductive probing element (pad) 6 is located on the posterior surface 17 of the substrate 15. The substrate 15 is preferably selected from the group consisting of a semiconductor wafer, a rigid polymer, a flexible polymer, glass, ceramic, or metal. The electrically conductive element 5 and 6 preferably includes a metal, doped region of semiconductor, or conducting polymer. The microneedle 10 comprises a protrusion of vertical extent of between 20 and 2000 μm from the base 19 of the substrate 15 and designed to penetrate a biological interface.

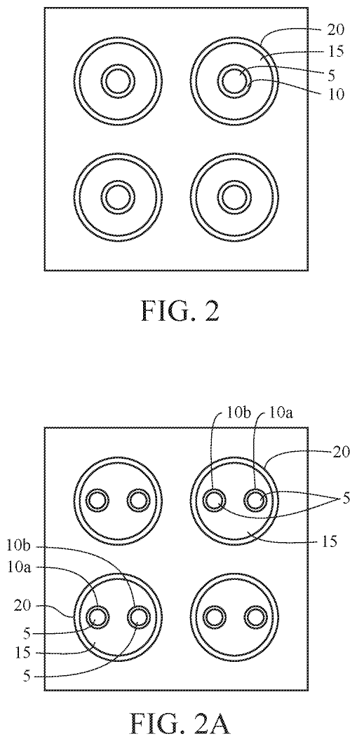

FIGS. 2 and 2A illustrate top plan views of the anterior surface 16 of the substrate 15 featuring the microneedle element 10 or elements 10a and 10b circumscribed by an insulating barrier 20.

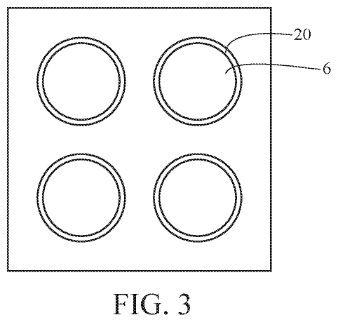

FIG. 3 is a view of the posterior surface 17 of the substrate 17 featuring the secondary electrically-conductive element (pad) 6 circumscribed by an insulating barrier 20.

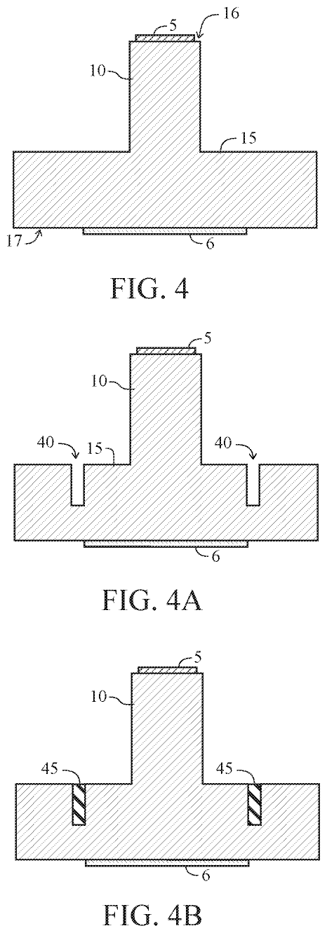

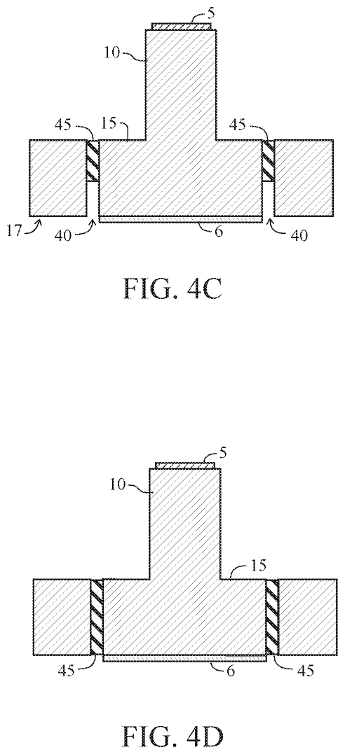

FIGS. 4, 4A, 4B, 4C and 4D illustrate a preferred fabrication process implemented to define insulating barrier regions to electrically isolate the microneedle and underlying substrate to facilitate electrical probing from the posterior surface of the substrate. As shown in FIG. 4, a substrate 15 has a microneedle 10 extending upward. A primary electrically-conductive sensing element 5 is located on an anterior surface 16 of the microneedle structure 1 and a secondary electrically-conductive probing element (pad) 6 is located on the posterior surface 17 of the substrate 15. As shown in FIG. 4, voids 40 are etched from the substrate 15. As shown in FIG. 4B, insulating material 45 fills the voids 40. As shown in FIG. 4C, voids 40 are etched from a posterior surface 17 of the substrate 15. As shown in FIG. 4D, insulating material 45 fills the voids 40 in the posterior surface 17 of the substrate 15.

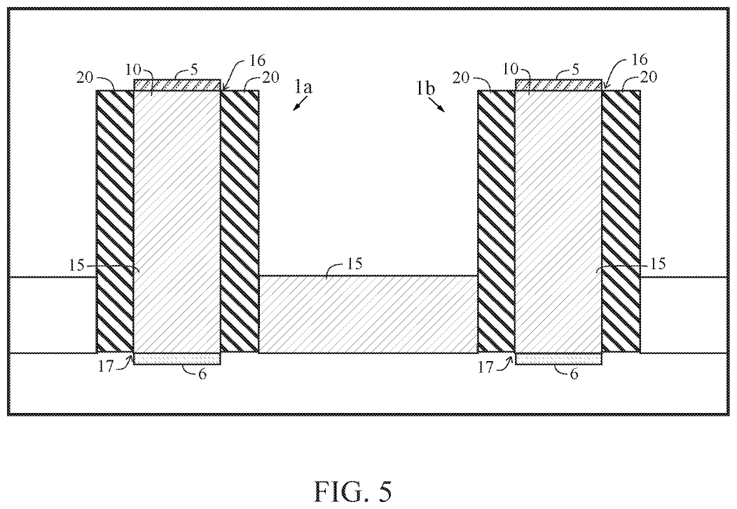

FIG. 5 is a cross-sectional view of a singular electrically-conductive microneedle structures 1a and 1b, electrically-isolated from one another, located on the anterior surface of an electrically-conductive substrate 15. Insulating barriers 20 are shown and possess a vertical extent equal to the sum of the thickness of the substrate 15 and the height of the microneedle 10. A primary electrically-conductive sensing element 5 is located on the distal extent of the microneedle 10 and a secondary electrically-conductive probing element (pad) 6 is located on the posterior surface 17 of the substrate 15, directly underneath the primary electrically-conductive sensing element 6.

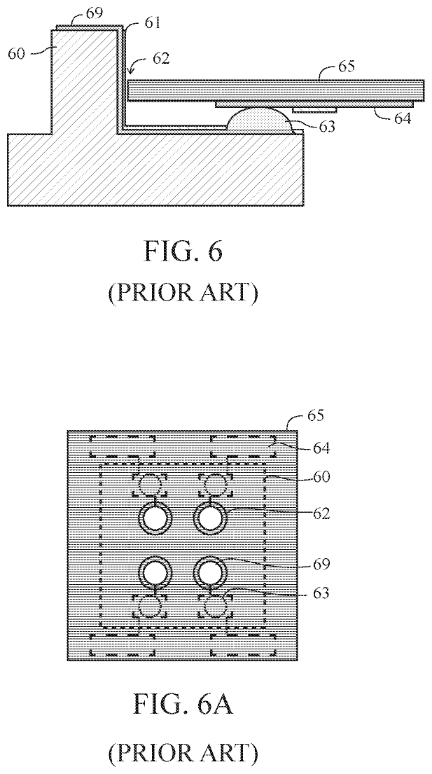

FIGS. 6 and 6A illustrate the prior art flip-chip bonding configuration and method of interface of a packaged microneedle sensing device. A substrate 60 has a metal surface trace 69, a surface insulation 61, an opening for a microneedle 62, a package substrate/housing 65, a flip chip bond 63 and an external package connection pad/connect 64.

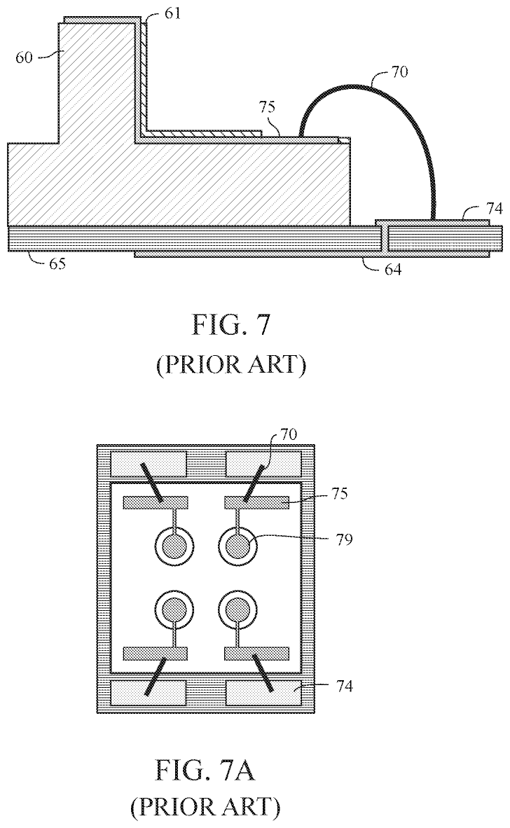

FIGS. 7 and 7A illustrate the prior art wire bonding configuration and method of interface of a packaged microneedle sensing device. A substrate 60 has a metal surface trace 75, a surface insulation 61, a package substrate/housing 65, a wire bond 70, an external package connection pad/connect 64, and an internal package contact 74.

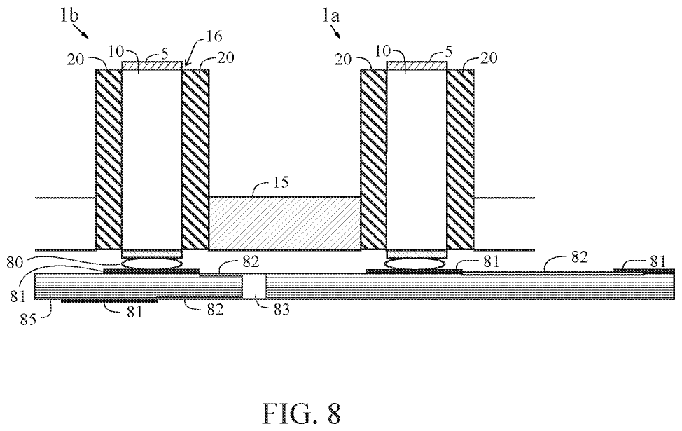

FIG. 8 is an illustration of a solder ball/stud bumping configuration and method of interface. Two electrically-isolated microneedle structures 1a and 1b occupying the same substrate 15, one structure 1b of which is mounted and interfaced using a via 83 between two opposing pads 81a and 81b to gain electrical access to the primary electrically-conductive element 5 on the posterior surface of the printed circuit board/package/chip carrier 85, and the other structure 1a of which is mounted and interfaced using two offset pads 81c and 81d connected by a trace 82 to gain electrical access to the primary electrically-conductive element 5 on the anterior surface 16 of the printed circuit board/package/chip carrier 85.

One embodiment is a microneedle-based electrochemical biosensors structure 1 with a printed circuit board (PCB) 85. The structure 1 comprises a PCB 85, a substrate 15, a microneedle biosensor 10, a primary electrically conductive element 5, a secondary electrically conductive element 6, an electrically insulative annular barrier 20 and a plurality of phase-change conductive interconnects. The phase-change conductive interconnect is preferably one of a solder ball 80 and a conductive epoxy. The PCB 85 comprises a pad 81, at least one via 83 and a plurality of traces 82. The substrate 15 is preferably composed of an electrically conducting material. The substrate preferably comprises an anterior surface 16 and a posterior surface 17. Each of the plurality of microneedle biosensors 10 has a length ranging from 20 microns to 2000 microns. Each microneedle biosensor 10 comprises a penetrating end and a posterior end. Each microneedle biosensor 10 protrudes from the anterior surface 16 of the substrate 15 and has a portion within the substrate 15. The primary electrically conductive element 5 is located on the penetrating end of each microneedle biosensor 10. The secondary electrically conductive element 6 is located on the posterior end of each microneedle biosensor 10. The electrically insulative annular barrier 20 surrounds the length of each microneedle biosensor 10 to electrically isolate the microneedle biosensor 10 from the substrate 15. Each of the phase-change conductive interconnects 80 is positioned between the pad 81 of the PCB 85 and the secondary electrically conductive element 6 of each microneedle biosensor 10.

The secondary electrically conductive element 6 is preferably composed of a metal, a doped region of semiconductor, or a conducting polymer.

Each microneedle biosensor 10 is preferably configured to penetrate a stratum corneum of a mammal.

The biological interface is preferably a stratum corneum.

The substrate 15 is preferably composed of a semiconductor wafer, a rigid polymer, a flexible polymer, a glass material, a ceramic material, or a metal material.
McCanna et al., U.S. patent application Ser. No. 14/843,926, filed on Sep. 2, 2015, for a Miniaturized Sub-Nanoampere Sensitivity Low-Noise Potentiostat System is hereby incorporated by reference in its entirety.
Windmiller et al., U.S. patent application Ser. No. 14/955,850, filed on Dec. 1, 2015, for a Method And Apparatus For Determining Body Fluid Loss is hereby incorporated by reference in its entirety.
Windmiller, U.S. patent application Ser. No. 15/177,289, filed on Jun. 8, 2016, for a Methods And Apparatus For Interfacing A Microneedle-Based Electrochemical Biosensor With An External Wireless Readout Device is hereby incorporated by reference in its entirety.
Wang et al., U.S. Patent Publication Number 20140336487 for a Microneedle Arrays For Biosensing And Drug Delivery is hereby incorporated by reference in its entirety.
Windmiller, U.S. patent application Ser. No. 15/590,105 for a Tissue-Penetrating Electrochemical Sensor Featuring A Co Electrodeposited Thin Film Comprised Of A Polymer And Bio-Recognition Element is hereby incorporated by reference in its entirety.
PCT Application Number PCT/US17/55314 for an Electro-Deposited Conducting Polymers For The Realization Of Solid-State Reference Electrodes For Use In Intracutaneous And Subcutaneous Analyte-selective Sensors is hereby incorporated by reference in its entirety.

From the foregoing it is believed that those skilled in the pertinent art will recognize the meritorious advancement of this invention and will readily understand that while the present invention has been described in association with a preferred embodiment thereof, and other embodiments illustrated in the accompanying drawings, numerous changes modification and substitutions of equivalents may be made therein without departing from the spirit and scope of this invention which is intended to be unlimited by the foregoing except as may appear in the following appended claim. Therefore, the embodiments of the invention in which an exclusive property or privilege is claimed are defined in the following appended claims.

1-17. (canceled)

18. A biosensor, comprising:
a substrate comprising a semiconductor;
a microneedle having a first portion within the substrate and a second portion extending from an anterior surface of the substrate, the second portion comprising a body and a penetrating end of the microneedle;
an electrical contact located on the penetrating end of the microneedle; and
an electrically insulative barrier surrounding the first portion of the microneedle and the body of the second portion of the microneedle,
wherein the electrically insulative barrier electrically isolates the microneedle from the substrate.

19. The biosensor of claim 18, wherein the electrical contact comprises a metal, a doped region of semiconductor, or a conducting polymer.

20. The biosensor of claim 18, further comprising a second electrical contact located on a posterior end of the microneedle.

21. The biosensor of claim 20, wherein the second electrical contact comprises a metal, a doped region of semiconductor, or a conducting polymer.

22. The biosensor of claim 20, wherein the electrically insulative barrier circumscribes the second electrical contact.

23. The biosensor of claim 18, wherein the microneedle is configured to penetrate a stratum corneum of a mammal.

24. The biosensor of claim 18, wherein the penetrating end is at a distal end of the microneedle and is separated from the anterior surface of the substrate by a length of the body.

25. The biosensor of claim 18, wherein the microneedle is formed from the semiconductor of the substrate.

26. A biosensor, comprising:
a substrate comprising a semiconductor;
a plurality of microneedles, each microneedle having a first portion within the substrate and a second portion extending from an anterior surface of the substrate, the second portion of each microneedle comprising a body and a penetrating end of the microneedle;
a plurality of electrical contacts, each electrical contact located on the penetrating end of a respective microneedle of the plurality of microneedles; and
a plurality of electrically insulative barriers, each electrically insulative barrier surrounding the first portion and the second portion of a respective microneedle of the plurality of microneedles,
wherein each of the plurality of electrically insulative barriers electrically isolates a respective microneedle from the substrate.

27. The biosensor of claim 26, wherein each of the plurality of electrical contacts comprises a metal, a doped region of semiconductor, or a conducting polymer.

28. The biosensor of claim 26, further comprising a plurality of additional electrical contacts, each additional electrical contact located on a posterior end of a respective microneedle of the plurality of microneedles.

29. The biosensor of claim 28, wherein each of the additional electrical contacts comprises a metal, a doped region of semiconductor, or a conducting polymer.

30. The biosensor of claim 28, wherein each of the electrically insulative barriers circumscribes a corresponding additional electrical contact of a respective microneedle.

31. The biosensor of claim 26, wherein each of the plurality of microneedles is configured to penetrate a stratum corneum of a mammal.

32. The biosensor of claim 26, wherein the penetrating end is at a distal end of each microneedle and is separated from the anterior surface of the substrate by a length of the body of each microneedle.

33. The biosensor of claim 26, wherein each microneedle of the plurality of microneedles is formed from the semiconductor of the substrate.

34. A biosensor, comprising:
a substrate comprising an electrically conducting material;
a microneedle within and extending from the substrate;
an electrical contact on a penetrating end of the microneedle; and
a barrier surrounding the microneedle within the substrate and along a length of the microneedle extending from the substrate, wherein the barrier electrically isolates the microneedle from the substrate.

35. The biosensor of claim 34, wherein the electrically conducting material of the substrate comprises a semiconductor, a rigid conducting polymer, a flexible conducting polymer, or a metal.

36. The biosensor of claim 34, wherein the electrical contact comprises a metal, a doped region of semiconductor, or a conducting polymer.

37. The biosensor of claim 34, further comprising a second electrical contact located on a posterior end of the microneedle.

38. The biosensor of claim 37, wherein the second electrical contact comprises a metal, a doped region of semiconductor, or a conducting polymer.

39. The biosensor of claim 37, wherein the barrier circumscribes the second electrical contact.

40. The biosensor of claim 34, wherein the microneedle is configured to penetrate a stratum corneum of a mammal.

41. The biosensor of claim 34, wherein the penetrating end is at a distal end of the microneedle and is separated from an anterior surface of the substrate by a length of a body of the microneedle.

42. The biosensor of claim 34, wherein the microneedle is formed from the electrically conducting material of the substrate.
